(19) United States
(12) Patent Application Publication
(10) Pub. No.: US 2021/0353146 A1
(43) Pub. Date: Nov. 18, 2021

(54) SYSTEM AND METHOD FOR PEOPLE WELLNESS MONITORING

(71) Applicant: Vikram Nathan, San Jose, CA (US)

(72) Inventor: Vikram Nathan, San Jose, CA (US)

(21) Appl. No.: 17/317,719

(22) Filed: May 11, 2021

Related U.S. Application Data
(60) Provisional application No. 63/023,259, filed on May 12, 2020.

(51) Int. Cl. A61B 5/00; A61B 5/0205; A61B 5/024; A61B 5/021; A61B 5/0533; A61B 5/1455

(52) U.S. Cl. CPC A61B 5/0022; A61B 5/02055; A61B 5/024; A61B 5/681; A61B 5/0533; A61B 5/14551; A61B 5/021

(57) ABSTRACT

The present invention provides a wellness monitoring system provided with one or more processors and a memory coupled to the one or more processors. The wellness monitoring system includes a first device, a system cloud server, and a second device. The first device is handled by a first person. The first device includes a routine monitoring application configured to collect a set of data associated with the first person. The system cloud server is connected with the first device and the second device through a communication network. The system cloud server collects and stores the set of data collected by the routine monitoring application. The second device is associated with a second person, wherein the second device is configured to review, analyze, and compare, the set of data stored at the system cloud server.

### CROSS REFERENCE TO RELATED APPLICATIONS

This application claims the benefit of U.S. Provisional Patent Application No. 63/023,259 filed on May 12, 2020 and titled A NEW SYSTEM FOR PEOPLE WELLNESS MONITORING REMOTELY USING MOBILE DEVICES by Vikram NATHAN, the entire disclosure of which is incorporated herein by reference.

### FIELD OF THE INVENTION

The invention is in the field of computer systems and, more specifically, related to wellness monitoring and analyzing daily routine of a person.

### BACKGROUND

The ability to adequately care for loved ones in an even more busy world, is a prevailing concern in today's society, particularly when considering the large number of “baby boomer” generation members rapidly approaching elder age. Further, unlike historical family units, it is increasingly common for loved ones, who are caregivers or support providers, to live in geographically disparate areas, sometimes even in different countries. Mobile consumer electronic devices, such as mobile phones, personal digital assistants (PDAs), portable gaming devices, and other electronic devices have become increasingly ubiquitous, and the functionality available to such devices continues to increase. The ever-present nature and ease of use of such devices has enabled geographically separated loved ones, caregivers, or support providers to communicate simply and easily.

However, the current systems and methods of obtaining remote wellness data include (i) calling the elder person; (ii) sending screen share image; (iii) or other manual methods of obtaining information about an elder person. Also, health trackers lack the ability to tracking wellness over a period of time (e.g., months or years). In addition, health trackers lack the ability to share insights for trending information with family or caregivers in an automated fashion. Health trackers lack the ability to allow family members and/or caregivers to subscribe to information about an elder person being monitored once the elder person has given the family members and/or caregivers permission. Therefore, what is needed is a system and method for improved health data sharing about an elder person with family members and/or caregivers.

### SUMMARY OF THE INVENTION

Disclosed is a system and method for improved health data sharing about an elder person with family members and/or caregivers. In an aspect, the present invention provides a wellness monitoring system provided with one or more processors and a memory coupled to the one or more processors. The wellness monitoring system includes a first device, a system cloud server, and a second device. The first device is handled by a first person. The first device includes a routine monitoring application configured to collect a set of data associated with the first person. The system cloud server is connected with the first device and the second device through a communication network. The system cloud server collects and stores the set of data collected by the routine monitoring application. The second device is associated with a second person, wherein the second device is configured to review, analyze, and compare, the set of data stored at the system cloud server.

In an embodiment of the present disclosure, the set of data comprising first person's wakeup time, sleeping time, a number to steps taken in given time, a number of distance moved in given time, calories or energy spent in given time, the first device usage or screen time, the first device application usage or breakdown or types of usages, and calling time.

In an embodiment of the present disclosure, the first device includes a mobile device, and wearable devices, such as, smart watch.

In an embodiment of the present disclosure, the wellness monitoring system calculates Heart rate, Pulse Oxygen level, Body temperature, Heart rhythms, ECG, Blood Pressure, and Galvanic skin response (GSR).

In an embodiment of the present disclosure, the second device displays the set of data in a plurality of forms. In addition, the plurality of forms includes charts, Graphics, Dashboards, Tables, multiple pages of data, and comparison table.

In an embodiment of the present disclosure, the wellness monitoring system captures an emotion state data of the first person and sends the emotion state data.

In an embodiment of the present disclosure, the second device includes a mobile device, a laptop, a personal computer, a tablet, and a wearable device.

In another aspect, the present invention provides a method for monitoring daily routine of the first person using a wellness monitoring system. The method includes a step to receive the method to receive a historic data of the first person. The historic data provides health pattern of the first person. The method includes another step to collect the set of data of the first person on daily basis via the first device. The method includes yet another step to store the set of data for the first person with facilitation of the system cloud server. The method includes yet another step to display the set of data via the second device. The wellness monitoring system extracts abnormal or unusual pattern of the first person based on a predetermined threshold set by comparing the historic data and the set of data received on daily basis. The wellness monitoring system automatically alerts the second person through the second device, in case of any unusual pattern observed during wellness monitoring.

### BRIEF DESCRIPTION OF THE DRAWINGS

The embodiments herein will be better understood in view of the description of the drawings which are as follows:

FIG. 1 illustrates an architecture for a wellness monitoring system, in accordance with various embodiments of the present disclosure;

FIG. 2 illustrates the wellness monitoring system 200, in accordance with various embodiments of the present disclosure;

FIG. 3 illustrates a flow chart depicting a method for monitoring daily routine of a first person using a wellness monitoring system, in accordance with various embodiments of the present disclosure;

FIG. 4 illustrates a hardware framework of the wellness monitoring system, in accordance with various embodiments of the present disclosure;

FIG. 5 illustrates a report for an elder person's wellness and activity metrics, in accordance with various embodiments of the present disclosure;

FIG. 6 illustrates a report for an elder person's wellness and activity metrics over a time period, in accordance with various embodiments of the present disclosure; and

FIG. 7 illustrates a report an elder person's interface, in accordance with various embodiments of the present disclosure.

### DETAILED DESCRIPTION

In the following description, for purposes of explanation, numerous specific details are set forth in order to provide a thorough understanding of the present technology. It will be apparent, however, to one skilled in the art that the present technology can be practiced without these specific details. In other instances, structures and devices are shown in block diagram form only in order to avoid obscuring the present technology.

Reference in this specification to “one embodiment” or “an embodiment” means that a particular feature, structure, or characteristic described in connection with the embodiment is included in at least one embodiment of the present technology. The appearance of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

Moreover, although the following description contains many specifics for the purposes of illustration, anyone skilled in the art will appreciate that many variations and/or alterations to said details are within the scope of the present technology. Similarly, although many of the features of the present technology are described in terms of each other, or in conjunction with each other, one skilled in the art will appreciate that many of these features can be provided independently of other features. Accordingly, this description of the present technology is set forth without any loss of generality to, and without imposing limitations upon, the present technology.

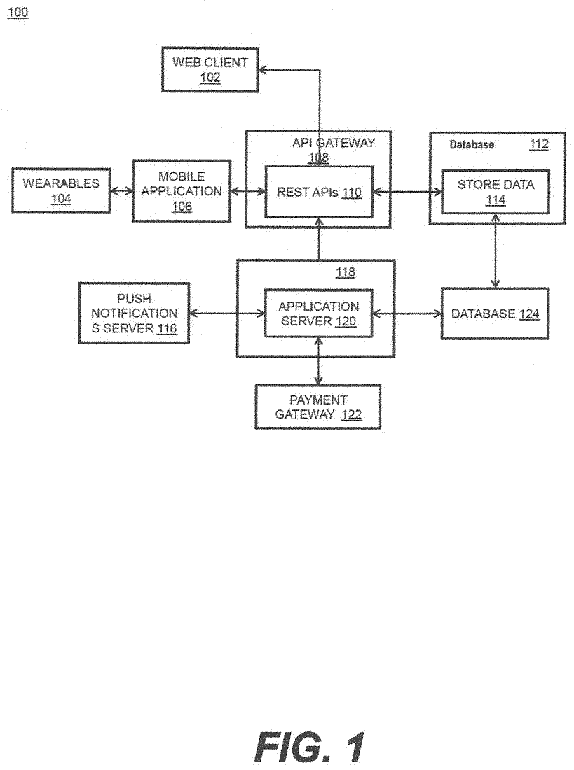

FIG. 1 illustrates an architecture 100 for a wellness monitoring system, in accordance with various embodiments of the present disclosure. The architecture 100 includes a web client 102, a wearable device 104, a mobile application 106, API gateway 108, Rest APIs 110, database 112, store data 114, Push notifications server 116, server 118, an application server 120, a payment gateway 122, and a database 124.

The architecture 100 includes the Web Client 102. In an embodiment of the present disclosure, the web client basically refers to the web browser of the architecture 100. In general, Web Client class facilitates sending data to or receiving data from any local, intranet, or Internet resource identified by a URI. In addition, Web Client class uses the Web Request class to provide access to resources.

The architecture 100 includes the wearable device 104. In an embodiment of the present disclosure, the wearable device 104 (e.g., smart watch). In an embodiment of the present disclosure, the wearable device 104 is embedded in human clothes, implanted in human body, and the like. The wearable device 104 includes the mobile application 106. In addition, the wearable device 104 facilitates the mobile application in monitoring daily health condition of a person.

The architecture 100 includes API gateway 108. In general, API Gateway is a service for creating, publishing, maintaining, monitoring, and securing REST, HTTP, and WebSocket APIs.

The architecture 100 includes REST APIs 110. In general, REST APIs is an application programming interface (API or web API) that conforms to the constraints of REST architectural style and allows for interaction with RESTful web services. In other words, REST APIs interact with a computer or system to retrieve information or perform a function. The REST APIs 110 is directly connected with the Web Client 102.

The architecture 100 includes database 112. In general, database 112 receives a command. The command is executed on store data 114 and the results is returned via database 112.

The architecture 100 includes push notification server 116. In general, Push notification server enables service of information to a computing device from an application server where the request for the transaction is initiated by the server rather than by an explicit request from the client.

The architecture 100 includes server 118. Server 118 may include one or more servers and may resize based on the workload.

The architecture 100 includes application server 120. In general, application server refers to server specifically designed to run applications. In addition, in general, “server” includes both the hardware and software that provide environment for programs to run.

The architecture includes payment gateway 122. In general, payment gateway captures and transfers payment data from customer to acquirer and then transfers payment acceptance or decline back to customer. In addition, payment gateway validates customer's card details securely, ensures the funds are available and eventually enables merchants to get paid. Further, payment gateway acts as an interface between a merchant's website and acquirer. Furthermore, payment gateway encrypts sensitive credit card details, ensuring that information is passed securely from the customer to acquiring bank, via merchant.

The architecture 100 includes the database 124. The database 124 is used for storage purposes. In general, database is a collection of information that is organized so that it can be easily accessed, managed and updated. In an embodiment of the present disclosure, the database 124 provides storage location to all data and information required by the architecture 100. In an embodiment of the present disclosure, the database 124 may be at least one of hierarchical database, network database, relational database, object-oriented database, and the like. However, the database 124 is not limited to the above-mentioned databases.

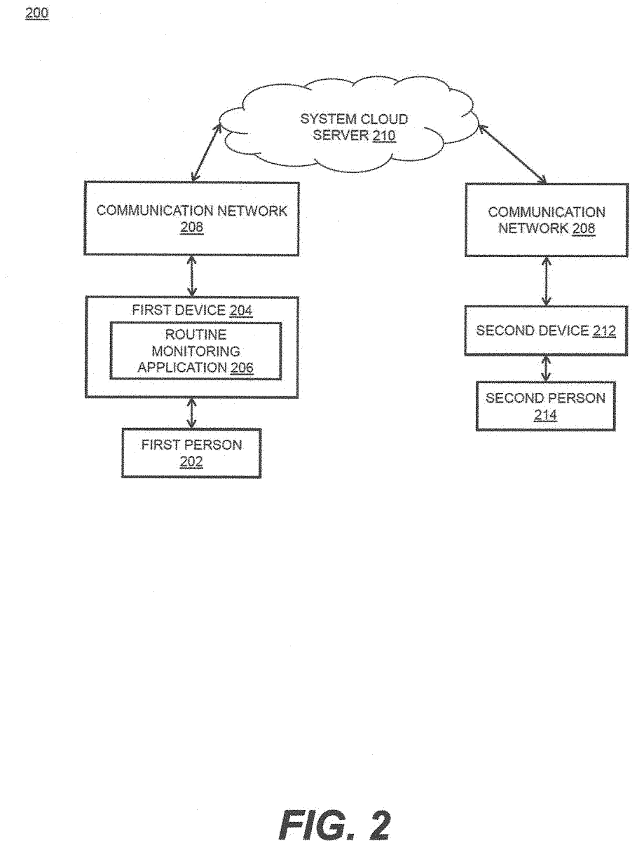

FIG. 2 illustrates the wellness monitoring system 200, in accordance with various embodiments of the present disclosure. The wellness monitoring system 200 includes a first person 202, a first device 204, a routine monitoring application 206, a communication network 208, a system cloud server 210, a second device 212, and a second person 214.

The wellness monitoring system 200 includes the first device 204. In an embodiment of the present disclosure, the first device 204 is handled by the first person 202. The first device 204 includes the routine monitoring application 206 configured to collect a set of data associated with the first person 202. In an embodiment of the present disclosure, the set of data includes but may not be limited to first person's wakeup time, sleeping time, a number to steps taken in given time, a number of distance moved in given time, calories or energy spent in given time, the first device usage or screen time, the first device application usage or breakdown or types of usages, and calling time. In an embodiment of the present disclosure, the first device includes but may not be limited to a mobile device, and wearable devices, such as, smart watch. In an embodiment of the present disclosure, the first person is the elder person that the wellness and activity is being monitored.

The wellness monitoring system 200 includes the system cloud server 210. In an embodiment of the present disclosure, the system cloud server 210 is connected with the first device 204 and the second device 212 through the communication network 208. The system cloud server 210 collects and stores the set of data collected by the routine monitoring application 206.

In an embodiment of the present disclosure, the wellness monitoring system uses a heath electronic device (e.g., a health watch) to acquire Heart rate, Pulse Oxygen level, Body temperature, Heart rhythms, ECG, Blood Pressure, and Galvanic skin response (GSR) via a wearable device on the first person. In an embodiment of the present disclosure, any health metric a health electronic device can measure may be incorporated into the wellness monitoring. In an embodiment of the present disclosure, the wellness monitoring system prompts an emotional state of the first person 202 and sends the first person 202 emotional state data to system cloud server 210. For example, the first person is prompted to input their emotional state as one of happy, OK, sad, fine, and lonely. After the first person selects “happy”, the first person's emotional state of “happy” is sent to system cloud server 210. In an embodiment of the present disclosure, daily living about the first person is prompted from the first person 202 and sent to system cloud server 210. For example, the first user 210 can be prompted about what and when they ate (e.g., salad at lunch), what activities have they participated in, what games did they play today, how much did they walked today, have read today (e.g., books, papers, internet articles, etc.), did socialize today, etc. In an embodiment of the present disclosure, the first person 202 emotional state may be estimated based on the first person 202.

The wellness monitoring system 200 includes second device 212. In an embodiment of the present disclosure, the second device 212 is associated with the second person 214. In an embodiment of the present disclosure, the second device 212 is configured to review, analyze, and compare, the set of data stored at the system cloud server 210. The second device 212 displays the set of data in a plurality of forms. The plurality of forms comprising charts, Graphics, Dashboards, Tables, multiple pages of data, and comparison table. In an embodiment of the present disclosure, the second device 212 includes a mobile device, a laptop, a personal computer, a tablet, and a wearable device. In an embodiment of the present disclosure, the second person 214 is a caregiver and/or family member.

The wellness monitoring system 200 includes the communication network 208. The communication network 208 provides medium to the first device 204 and the second device 212 to connect with the system cloud server 210. The communication network 208 use protocols to connect the system cloud server 210 with the first device 204 and the second device 212. In an embodiment of the present disclosure, the communication network 208 facilitates transmission of the set of data to the system cloud server 210. In general, communication network is associated with hardware devices that are capable of transmitting data.

The communication network 208 provides network connectivity to the system cloud server 210 using a plurality of methods. The plurality of methods is used to provide network connectivity to the health maintenance facility 102 include 2G, 3G, 4G, Wi-Fi, BLE, LAN, VPN, WAN, and the like. In an example, the communication network 208 includes but may not be limited to a local area network, a metropolitan area network, a wide area network, a virtual private network, a global area network and a home area network.

In an embodiment of the present disclosure, the communication network 208 is any type of network that provides internet connectivity to the system cloud server 210. In an embodiment of the present disclosure, the communication network 208 is a wireless mobile network. In another embodiment of the present disclosure, the communication network 208 is a wired network with a finite bandwidth. In yet another embodiment of the present disclosure, the communication network 208 is combination of the wireless and the wired network for optimum throughput of data transmission. In yet another embodiment of the present disclosure, the communication network 208 is an optical fiber high bandwidth network that enables high data rate with negligible connection drops.

In an example, routine monitoring application in the first device collects data about motion and accelerator and sensor data and analyzes the movement pattern about the first person. In addition, the first person refers to elder people, who requires more care. In an example, the set of data includes data frequency of, every 1 hour or 15 minutes or 1 minute, or even every 4 hours or 6 or 12 or 24 hours. In addition, these data sharing frequency can be adjusted and configurable per customer needs.

In an example, the system cloud server can serve world-wide and collect the set of data from anyone anywhere in the world and at any time and at any frequency. In addition, the system cloud server can also connect with multiple users, can be 1000 or 1 million or even 1 billion or more if required.

In an example, the set of data is stored in a local database data structure. In addition, these database data are stored by user or mobile device, time, date and type of data. Further, these may be stored in relational tables or databases or non-relational database or non-structured databases or even object-oriented databases. Furthermore, searches can be performed on the set of data, reporting and queries may be performed on the set of data based on user, date, time, and the like.

In an example, the second device is handled by the second person. In addition, the second person is a user, who is reviewing, analyzing and benefiting from the set of data about elder people. In an embodiment of the present disclosure, the second person is a family members, sons, daughters, spouse, nurses or caregivers or doctors or any other interested parties or professionals or personal friends. Furthermore, the second person is granted access to receive the set of data every few minutes or hours or daily. Moreover, the set of data including dashboards or charts, graphics and wellness data are provided regularly in multiple formats. Also, the set of data is simply display or table or charts or reports. Also, the set of data recorded includes data of today, daily, last week or last month or last year or over a period of time.

In an example, the set of data recorded is compared, contrasted against the historic data. In addition, the set of data may also be trended against to see any insights or issues, abnormalities or trends or degradation in the data or movement or patterns. Further, the set of data may be displayed as charts or graphics or dashboards.

In an example, the second device is configured to perform comparison and analytics of the set of data recorded for the first person. In another related example, the set of data from the first person can be compared against a similar or different cohort or others based on age, location, gender, disease conditions. In addition, the set of data may be analyzed for trending such as past trends and latest changes such as person walking less daily. Further, Data Alerts can be gathered and sent to the second person, for example, in case the first person usually gets up at 6 am and not up yet today till 9 am. Furthermore, various analytics, statistics, calculations, mathematics formulas or algebraic methods or machine learning, deep learning neural networks, Artificial Intelligence or AI techniques or CNN or RNN, LSTM or other methods may be applied to analyze the set of data.

In an example, notifications and alerts about the first person data may be sent to the second person. In addition, notifications and alerts may be detecting and sending texts or notifications or emails and other forms of communicating with others. Further, these alerts may be abnormal or semi-normal. Furthermore, these events may be sent at given time or intervals. Moreover, these events may be elevated if one caregiver is not responding.

In an example, the wellness monitoring system can also capture an elder's emotional state like Happy, sad, normal, and the like. In addition, these states or info may be sent to the second person.

In an example the wellness monitoring system may also send daily or weekly or multiple times daily or monthly, or any frequency inspiring quotes, morale boosters, emotion boosters or positive messages to keep the first person happy. In addition, these quotes may be pre-programmer or sent by family members or caregivers.

In an example, with the permission of the first person, GPS, Location data can be gathered and reported as required by the second person. In addition, Location and how long, where, when and if not, all can be given to the second person with permissions. Hence, staying home or shopping or friends place or work or church can be determined and stored.

In an example, the wellness monitoring system can work and works across all regions, countries and or time zones and or any languages.

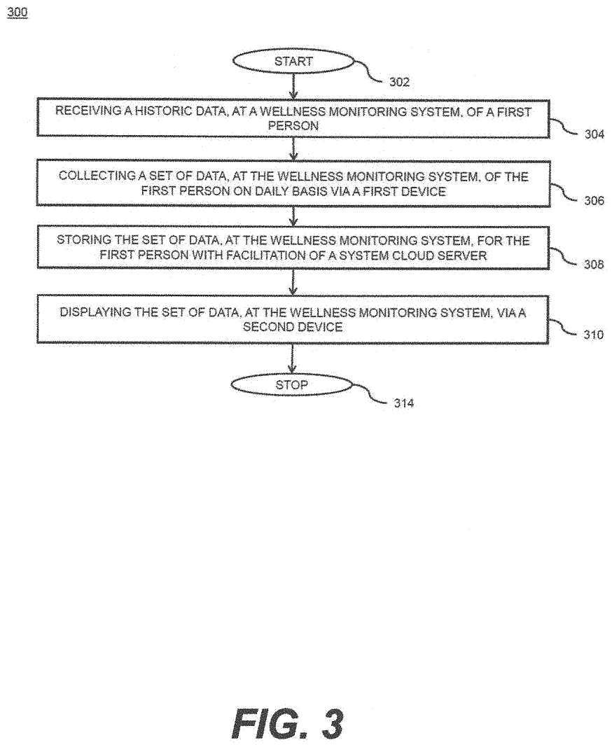

FIG. 3 illustrates a flow chart 300 depicting a method for monitoring daily routine of the first person 202 using the wellness monitoring system 200, in accordance with various embodiments of the present disclosure. The Flow chart 300 initiates at step 302, following at step 304, the method to receive a historic data of the first person 202. The historic data provides health pattern of the first person 202. At step 306, the method includes another step to collect the set of data of the first person 202 on daily basis via the first device 204. At step 308, the method includes yet another step to store the set of data for the first person 202 with facilitation of the system cloud server 210. At step 310, the method includes yet another step to display the set of data via the second device. In an embodiment of the present disclosure, the wellness monitoring system extracts abnormal or unusual pattern of the first person 202 based on a predetermined threshold set by comparing the historic data and the set of data received on daily basis. The wellness monitoring system automatically alerts the second person through the second device, in case of any unusual pattern observed during wellness monitoring. The flow chart terminates at step 312.

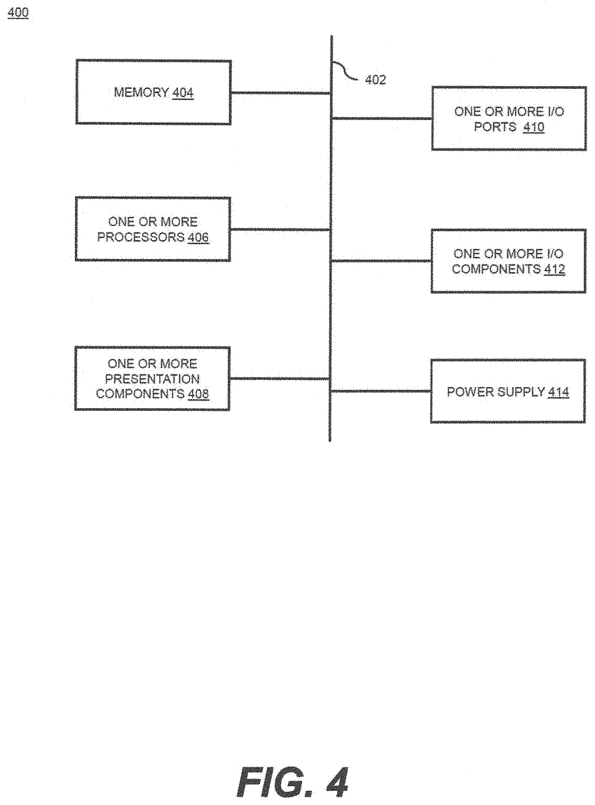

FIG. 4 illustrates a hardware framework 400 of the wellness monitoring system 200, in accordance with various embodiments of the present disclosure. The hardware framework 400 is required to run the network traffic analyzing system. The hardware framework 400 includes various components that work synchronously to enable processing of the network traffic analyzing system and allows storing of data in the network traffic analyzing system. The hardware framework 400 includes a bus 402 that directly or indirectly couples the following devices: memory 404, one or more processors 406, one or more presentation components 408, one or more input/output (I/O) ports 410, one or more input/output components 412, and an illustrative power supply 414. The bus 402 represents what may be one or more busses (such as an address bus, data bus, or combination thereof). Although the various blocks of FIG. 4 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. The inventors recognize that such is the nature of the art and reiterate that the diagram of FIG. 4 is merely illustrative of an exemplary hardware framework 400 that can be used in connection with one or more embodiments of the present invention. Distinction is not made between such categories as “workstation,” “server,” “laptop,” “hand-held device,” etc., as all are contemplated within the scope of FIG. 4 and reference to “hardware framework.”

The hardware framework 400 typically includes a variety of computer-readable media. The computer-readable media can be any available media that includes both volatile and nonvolatile media, removable and non-removable media. By way of example, and not limitation, the computer-readable media may comprise computer storage media and communication media. The computer storage media includes volatile and nonvolatile, removable and non-removable media implemented in any method or technology for storage of information such as computer-readable instructions, data structures, program modules or other data. The computer storage media includes, but is not limited to, non-transitory computer-readable storage medium that stores program code and/or data for short periods of time such as register memory, processor cache and random access memory (RAM), or any other medium which can be used to store the desired information. The computer storage media includes, but is not limited to, non-transitory computer readable storage medium that stores program code and/or data for longer periods of time, such as secondary or persistent long term storage, like read only memory (ROM), EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information. The communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 404 includes computer-storage media in the form of volatile and/or nonvolatile memory. The memory 404 may be removable, non-removable, or a combination thereof. Exemplary hardware devices include solid-state memory, hard drives, optical-disc drives, etc. The hardware framework 400 includes the one or more processors 406 that read data from various entities such as memory 404 or I/O components 412. The one or more presentation components 408 present data indications to a user or other device. Exemplary presentation components include a display device, speaker, printing component, vibrating component, etc.

It is to be understood, however, that even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms mentioned.

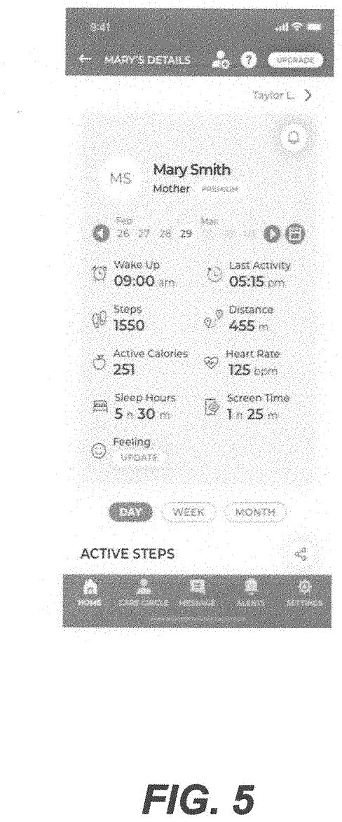

FIG. 5 illustrates a report for an elder person's wellness and activity metrics, in accordance with various embodiments of the present disclosure. In an embodiment of the present disclosure, the wellness and activity metrics can be shared, in full or in part, with caregivers, family, friends, and any other person the elder person would like to share with. In an embodiment of the present disclosure, the wellness and activity metrics are shown in real time to another person (e.g., caregiver, family, friends, etc.). For example, when a metric changes, the elder's application may notify a server of the change and the server can push an update to another person.

In an embodiment of the present disclosure, the wellness and activity metrics may include time went to bed, wake up time, sleep time and/or duration, waking hours and/or time awake, time of last activity (e.g., time since last walk), activity type, number steps the elder has taken, the distance the elder has walked, the calories the elder has burned, the average and/or instant heart rate, previous night sleeping time, time spent on electronic devices and/or last time using electronic device, emotional state, how close to any set goals, and any other metric related to wellness and/or activity. In an embodiment of the present disclosure, the wellness metrics may be shown for a single day and a range of time (e.g., week, month, year, user selectable range, etc.). In an embodiment of the present disclosure, the frequency that metrics are measured can vary (e.g., every second, every 5 seconds, every 10 seconds, etc.). In an embodiment of the present disclosure, metrics are stored on the elder's device and uploaded to a server (e.g., cloud storage). In an embodiment of the present disclosure, once the data is moved to a server, the data is deleted from the elder's device. In an embodiment of the present disclosure, another person (e.g., caregiver, family, friend, etc.) requests access to the data and is approved by the elder. In an embodiment of the present disclosure, another person (e.g., caregiver, family, friend, etc.), is invited to view the elder's data (e.g., elder invites person). In an embodiment of the present disclosure, parameters are collected from an electronic device (e.g., a watch, cellphone, etc.) and the parameters are used to calculate metrics. The metrics are sent to a remote server (e.g., a cloud server, database server, etc.). In an embodiment of the present disclosure, the parameters may be sent to a remote server (e.g., a cloud server) and the remote server may calculate the metrics.

In an embodiment of the present disclosure, alerts may be created for the elder person based on metrics. For example, walking outside their normal locations, high and/or low heartbeat, less activity, change in sleep patterns, moving less, etc.). In an embodiment of the present disclosure, each other person may configure a threshold for a given metric to trigger an alert. For example, one child may want to be notified if the elder has not gotten out of bed in the morning and another child may want to be notified if the elder has not go to bed by a certain time. In an embodiment of the present disclosure, an elect may be normal (e.g., the elder is within the historical normal range, OK, normal status, etc.) and abnormal (e.g., the elder is outside the historical normal range). In an embodiment of the present disclosure, another person can specify the criteria for when an alert is generated (e.g., less than 500 steps per day).

In an embodiment of the present disclosure, goals may be set by either the elder or another person (e.g., caregiver, family, friend, etc.). For example, a goal can be to wake up by 6 am daily and another goal to walk 2 miles per day. The metrics may show the status in relation to the goal. Alerts may be given when goals are not being obtained or not on track to be obtained. When goals are not being met, the system may notify the user to encourage them to obtain the goal.

In an embodiment of the present disclosure, the wake-up time may be determined by monitoring the accelerometer and/or gyro rotation sensor. Activity is measured by monitoring accelerometer and/or gyro rotation sensor. Based on the level of activity and threshold and levels of movements, then wake up time is determined. In an embodiment of the present disclosure, time the elder went to sleep may be estimated based monitoring accelerometer and/or gyro rotation sensor for movement and threshold and levels of movements. In an embodiment of the present disclosure, sleep time may be determined based on non-activity for a time period. In an embodiment of the present disclosure, sleep time may be determined based typically sleep hours of elder and non-activity indicators. For example, when no activity is detected for 1-2 hours the typical sleeping hours, then when the person went to sleep is determined based on last seen motion. For another example, when there is no motion for a long duration (e.g., 1 hr or 30 mins), the elder is determined to be asleep.

In an embodiment of the present disclosure, last seen activity is determined based on movement (e.g., accelerometer and/or gyro rotation sensor for movement and threshold and levels of movements). For example, system has not detected movement above a threshold for 30 minutes, the last seen time is 30 minutes before the present time.

In an embodiment of the present disclosure, a single person (e.g., caregiver, family member, etc.) may monitor multiple elders. In an embodiment of the present disclosure, multiple persons (e.g., caregiver, family member, etc.) may monitor multiple elders.

In an embodiment of the present disclosure, games, cognitive and memory tests may be used to generate wellness metrics. In an embodiment of the present disclosure, Geo fencing may be defined for an elder and alerts generated when elder leaves the geo fence area. In an embodiment of the present disclosure, geo fencing can include location names (e.g., room, park, main hall, out walking, friends place, etc.) and these names can be used as a metric. In an embodiment of the present disclosure, time socializing (e.g., friends, family, grandkids, etc.) may be used as a metric. In an embodiment of the present disclosure, when the elder indicates they are lonely, social interactions may be planned by another person (e.g., care giver, family, friends, etc.).

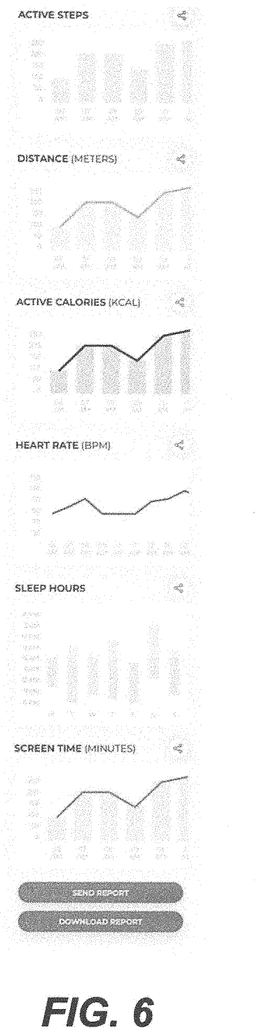

FIG. 6 illustrates a report for an elder person's wellness metrics over a time period, in accordance with various embodiments of the present disclosure. In an embodiment of the present disclosure, the wellness metrics may include wake up time, time of last activity, number steps the elder has taken, the distance the elder has walked, the calories the elder has burned, the average and/or instance heart rate, previous night sleeping time, time spent on electronic devices, emotional state, and any other metric related to wellness. In an embodiment of the present disclosure, the wellness metrics may be shown for a single day and a range of time (e.g., week, month, year, user selectable range, etc.). In an embodiment of the present disclosure, the report may be shared with another person, exported in another format, and downloaded.

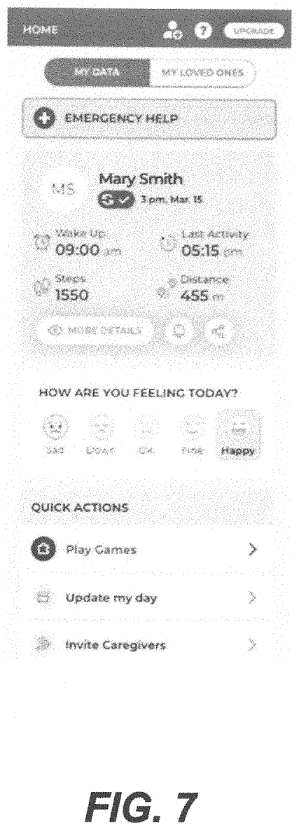

FIG. 7 illustrates a report an elder person's interface, in accordance with various embodiments of the present disclosure. In an embodiment of the present disclosure, the elder may view their metrics, click to request help, play games, set their emotional state, invite caregivers, and various other tasks.

What is claimed is:

1. A wellness monitoring system provided with one or more processors and a memory coupled to the one or more processors, wherein the wellness monitoring system comprising:
measuring one or more health parameters;
calculating one or more health and activity metrics based on health parameters; and
transmitting the one or more health and activity metrics.
